(19) United States
(12) Patent Application Publication
(10) Pub. No.: US 2008/0206331 A1
(43) Pub. Date: Aug. 28, 2008

(54) TABLET COMPRISING EFLETIRIZINE AND PSEUDOEPHEDRINE

(75) Inventors: Monique Berwaer, Ham-Sur-Heure-Nalinnes (BE); Anthony Guichaux, Bruxelles (BE); Serge Cuypers, Baisy-Thy (BE); Michel Deleers, Linkebeek (BE); Domenico Fanara, Wanze (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB, S.A., Brussels (BE)

(21) Appl. No.: 11/762,867

(22) Filed: Jun. 14, 2007

Related U.S. Application Data
(63) Continuation of application No. 10/537,553, filed on Jan. 17, 2006, now abandoned.

(30) Foreign Application Priority Data
Dec. 6, 2002 (EP) 02080127.0

(51) Int. Cl. A61K 9/22; A61K 9/20; A61K 31/495

(52) U.S. Cl. 424/468; 514/255.04; 424/464

(57) ABSTRACT

The present invention concerns a tablet comprising two distinct segments. More particularly the invention relates to combinations of two pharmaceutical substances and methods of treatment of allergic disorders.

The present invention concerns a tablet comprising two distinct segments. More particularly the invention relates to combinations of two pharmaceutical substances and methods of treatment of allergic disorders.

2-{2-[4-[bis(4-fluorophenyl)methyl)-1-piperazinyl]ethoxy}acetic acid or efletirizine, in the form of its dihydrochloride salt has the following formula:

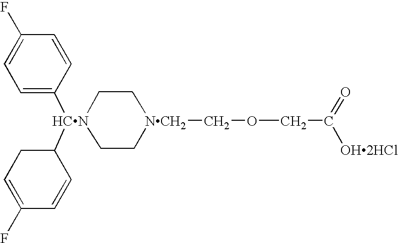

Efletirizine is encompassed within the general formula of European Patent No. 0 058 146 and may be prepared according to the general process described in this patent. Said process for the synthesis of 2-{2-[4-(diphenylmethyl)-1-piperazinyl]ethoxy}acetic acid derivatives comprises reacting a 1-(diphenylmethyl) piperazine derivative with methyl(2-chloroethoxy)acetate or 2-(2-chloroethoxy) acetamide to form a methyl 2-{2-[4-(diphenylmethyl)-1-piperazinyl]ethoxy}-acetate or a 2-{2-[4-(diphenylmethyl)-1-piperazinyl]ethoxy}acetamide, respectively. Thus the formed methyl ester or acetamide is then subjected to basic hydrolysis followed by acidification and isolation of the free carboxylic acid which is then transformed into its dihydrochloride.

European Patent No 1 034 171 describes two pseudo-polymorphic forms of efletirizine.

Efletirizine has been found to possess excellent antihistaminic properties. It belongs to the pharmacological class of second generation histamine H₁-receptor antagonists and shows in vitro high affinity and selectivity for H₁-receptors. It is useful as an antiallergic, antihistaminic, bronchodilator and antispasmodic agent, and also for the treatment of allergic rhinitis and rhino-conjunctivitis.

On the other hand, a compound pseudoephedrine, is well known as sympathomimetic drug recognised as safe therapeutic agents effective in the relief of nasal congestion.

It is well known to those skilled in the art that combinations of pharmaceutical substances should always be handled with care because they are very susceptible of inducing unpredictable adverse effects in humans. In some cases, they also induce an efficiency of the treatment which is lower than that of each pharmaceutical substance taken alone.

In the treatment of allergic disorders such as for example a pollen associated allergic rhino-conjunctivitis, care should be taken, when combining an antihistaminic and a decongestant, not only to increase the overall efficiency of the treatment, i.e. the percentage of days during the whole treatment period, when the symptoms of sneezing, rhinorrhea, nasal obstruction, lacrimation, nasal and ocular pruritus are absent or at the most mild, but also to avoid possible adverse effects like insomnia and headache.

Several patent applications already disclosed binary and/or ternary combinations of pharmaceutical substances in specific amounts in view of treating various disorders in humans. In particular United Kingdom Patent 2 311 940 and European patent application 0 811 374 disclose a pharmaceutical composition comprising cetirizine and pseudoephedrine; U.S. Pat. No. 6,171,618 discloses a dosage form containing cetirizine as an immediate release component and pseudoephedrine as a controlled release component, a portion of the pseudoephedrine can be incorporated as an immediate release component.

In a more particular way, the international patent application WO 98/41194 discloses a pharmaceutical composition which can be administered orally, allowing the immediate release of a first active substance and the prolonged release of the same or of a second active substance, comprising
A. at least one layer comprising an active substance and excipients which allow immediate release of the said active substance after administration, and
B. at least one second layer which allows the controlled release of the same or of a second active substance, this layer being a pharmaceutical composition comprising between 5 and 60% by weight, relative to the total weight of the composition, of at least one excipient, selected from inert matrices, hydrophilic matrices, lipid matrices, mixtures of inert matrices and of lipid matrices, mixtures of hydrophilic matrices and of inert matrices; and between 5 and 50% by weight, relative to the total weight of the composition, of at least one alkalinizing agent soluble in an aqueous phase under physiological pH conditions.

Due to the presence of the alkalinizing agent, this composition has demonstrated a good stability profile.

It has now surprisingly been found that such a pharmaceutical composition can be prepared by adding less than 5% of alkalinizing agent or in the absence of alkalinizing agent.

Despite the fact that a lower amount of alkalinizing agent has been added, the tablet of the invention has also demonstrated a good stability profile.

Thus an objective of the present invention is to provide a useful combination of pharmaceutical substances for treating various disorders in humans, said combination being able to increase the efficiency of said treatment over the efficiency of each substance alone, while avoiding adverse effects during the said treatment.

Another objective of the present invention is to provide such a useful combination of pharmaceutical substances when the treatment in question is a therapy such as needed for rhinitis, cold, flu, cold-like and flu-like symptoms.

The present invention encompasses a method of treating a disorder selected from rhinitis, cold, flu, cold-like and flu-like symptoms in a human, which comprises administering to a human in need of such therapy, a tablet comprising an effective amount of pseudoephedrine, an individual optical isomer or a pharmaceutically acceptable salt thereof and an effective amount of efletirizine or a pharmaceutically acceptable salt thereof.

The term “a method for treating a disorder selected from rhinitis, cold, flu, cold-like and flu-like symptoms in a human” as used herein means providing relief from the symptoms of sneezing, rhinorrhea, nasal obstruction, nasal and ocular pruritus, lacrymation, and the like.

The term “pharmaceutically acceptable salts” as used herein with respect to efletirizine means not only their addition salts with non-toxic organic and inorganic acids, such as acetic, citric, succinic, ascorbic, hydrochloric, hydrobromic, sulfuric, and phosphoric acids and the like, but also their metal salts (for example sodium or potassium salts), ammonium salts, amine salts and aminoacid salts.

The term “pharmaceutically acceptable salt” as used herein with respect to pseudoephedrine means namely its hydrochloride and sulfate and equivalent non-toxic salts.

The term “individual optical isomer” as used herein means, when the molecule has a centre of asymmetry, the levorotatory and the dextrorotatory enantiomers thereof. As is well known in the art, purification of such enantiomers is a rather difficult process depending upon the selected way of preparation of the compound and the optical purity of the starting material. Therefore the term “individual optical isomer” as used herein means that the said compound comprises at least 90%, preferably at least 95%, by weight of the said individual (either dextro- or levorotatory) optical isomer and at most 10%, preferably at most 5%, by weight of the other individual (respectively levo- or dextrorotatory) optical isomer. Additionally, each individual optical isomer can be prepared from the racemic mixture by enzymatic biocatalytic resolution, such as disclosed in U.S. Pat. Nos. 4,800,162 and 5,057,427.

The preferred compounds for efletirizine are the acid and its dihydrochloride salt.

In the present application the term “pseudoephedrine”, used herein means pseudoephedrine itself, an individual optical isomer or a pharmaceutically acceptable salt thereof.

In the present application the term “efletirizine” means efletirizine itself (2-{2-[4-[bis(4-fluorophenyl)methyl)-1-piperazinyl]ethoxy}acetic acid), or a pharmaceutically acceptable salt thereof.

In a particular embodiment, the present invention concerns a tablet comprising at least two distinct segments, one segment of which comprises as active ingredient predominantly efletirizine and a second segment of which comprises as active ingredient predominantly pseudoephedrine, said segments being composed and formed in such a way that the resulting tablet is substantially free of impurities formed by reaction of efletirizine with pseudoephedrine and with the proviso that the tablet comprises less than 5% by weight, relative to the total weight of the pseudoephedrine segment, of an alkalinizing agent. Indeed, it was demonstrated that if efletirizine and pseudoephedrine were formulated together in the same segment, a degradation of efletirizine appeared due to a chemical reaction with pseudoephedrine.

In a second embodiment, the invention concerns a tablet comprising at least two distinct segments one segment of which comprises as active ingredient predominantly efletirizine and a second segment of which comprises as active ingredient predominantly pseudoephedrine, said segments being composed and formed in such a way that the pharmacokinetic profiles of the efletirizine and pseudoephedrine are substantially the same as in a dosage form containing each as sole active ingredient in the same amount.

By the term “segment” we understand a discrete volume of a pharmaceutical composition containing an active drug and one or more pharmaceutically acceptable excipients. A segment of a tablet may form, for example, a layer of a multilayer tablet (i.e. a layer of a bilayer tablet) or a core of a tablet or a coating fully or partially covering a core of a tablet. A segment may also be a particle fully or partially covered by a coating or a coating fully or partially covering a particle.

By “substantially free” we understand less than 5%, preferably less than 3% by weight. More preferably we understand less than 0.5%, further more less than 0.2% by weight.

Preferably, in the tablet according to the invention, the pseudoephedrine segment is substantially free of efletirizine, by which is meant less than 5%, preferably less than 3%, more preferably less than 0.5% of the efletirizine segment content in the pseudoephedrine segment. Preferably, in the tablet according to the invention, the efletirizine segment is substantially free of pseudoephedrine, by which is meant less than 5%, preferably less than 3%, more preferably less than 0.5% of the pseudoephedrine segment content in the efletirizine segment.

In another embodiment of the invention the tablet further comprises a barrier segment wherein said barrier segment separates the efletirizine segment and the pseudoephedrine segment. The barrier segment comprises materials known to persons skilled in the art.

In another embodiment of the invention, the pseudoephedrine segment comprises less than 5% by weight, relative to the total weight of the pseudoephedrine segment, of an alkalinizing agent.

The alkalinizing agent which can be used according to the present invention should preferably be soluble in the aqueous phase under physiological pH conditions. The alkalinizing agent may be chosen from alkali or alkaline-earth metal hydroxides, carbonates, bicarbonates and phosphates, sodium borate as well as basic salts of organic acids (example: sodium citrate). On the other hand, salts not soluble in water under physiological pH conditions, such as dibasic calcium phosphate, are not suitable according to the present invention.

In another embodiment of the invention, the tablet comprises a plurality of pseudoephedrine segments.

Preferably the efletirizine segment of the tablet is in the form of a compression coating or alternatively in the form of a spray coating. By the term “compression coating” we understand a small tablet utilized as part of the compression of a second tablet and where the small tablet is located almost in the centre and the rest of the powder compressed outside. By the term “spray coating” we understand an over coating of a tablet with the coating preparation containing an active substance.

Preferably the pseudoephedrine segment of the tablet contains inert pharmaceutical excipients in an amount of 0.75 to 4.5 times that of the pseudoephedrine itself by weight, and more preferably of 1 to 3 times.

Preferably the efletirizine segment of the tablet contains inert pharmaceutical excipients in an amount of 5 to 30 times that of the efletirizine itself by weight, and more preferably of 10 to 20 times.

Preferably the ratio of the total amount of inert pharmaceutical excipients present to the total aggregate amount of all active ingredients is between 1.2 and 6 by weight. The best results have been obtained with a ratio of about 3.

In a preferred b.i.d. (b.i.d.=twice a day) tablet according to the invention the weight ratio of pseudoephedrine to efletirizine is between 2 and 40. The best results have been obtained with a ratio of about 12.

In a more preferred b.i.d. tablet the pseudoephedrine segment comprises about 108 to 160 mg, preferably 90 to 150 mg and more preferably 120 mg of pseudoephedrine and the efletirizine segment comprises about 3 to 25 mg and preferably 15 mg of efletirizine.

In that case, according to the invention, the interfacial surface area of the pseudoephedrine segment and efletirizine segment is less than 180 mm², and preferably from about 20 to about 150 mm². By interfacial area we understand the calculated contact surface between the two segments what ever the type of tablet (round, oblong, squared, caplet, . . . ) or the type of contact could be.

In a preferred embodiment of the invention the pseudoephedrine segment is a slow release formulation. By “slow release”, we understand a release of 10 to 60% in 1 hour, and greater than 70% in 6 hours, or 40 to 80% in 2 hours, and greater than 70% in 6 hours in 500 ml water (HCl 0.1N) in USP apparatus 1 (37° C., 100 RPM).

In a preferred embodiment of the invention the efletirizine is in immediate release form. By “immediate release” we understand a release of more than 70% in 30 minutes, in 500 ml water (HCl 0.1N) in USP apparatus 1 (37° C., 100 RPM).

The b.i.d tablet weight is between 200 to 800 mg, and preferably between 300 and 600 mg.

In a preferred once a day tablet the pseudoephedrine segment comprises about 90 to 265 mg of pseudoephedrine and the efletirizine segment comprises about 15 to 70 mg of efletirizine and parameters of this tablet (for example interfacial area between segments, weight limits of the tablet, . . . ) have to be adapted by persons skilled in the art.

Preferably the tablet according to the invention comprises an amount of efletirizine which when dosed to a human subject gives a efletirizine area under the efletirizine plasma concentration versus time curve which is between 80% and 125% of the area under the efletirizine plasma concentration versus time curve observed when a dihydrochloride efletirizine immediate release tablet comprising said amount of efletirizine is dosed to same human subject at the same efletirizine dose.

Preferably the tablet according to the invention comprises an amount of pseudoephedrine which when dosed to a human subject gives a pseudoephedrine area under the pseudoephedrine plasma concentration versus time curve which is between 80% and 125% of the area under the pseudoephedrine plasma concentration versus time curve observed when a pseudoephedrine sustained release tablet comprising said amount of pseudoephedrine is dosed to same human subject.

Pseudoephedrine/efletirizine dosage forms of this invention provide pseudoephedrine and efletirizine blood or plasma levels which are equivalent to those resulting from dosing separate pseudoephedrine and efletirizine control formulation.

In the tablet according to the invention the particle size of the pseudoephedrine present is chosen such that it has a flow index less than 25. By “flow index” we understand the flowability index corresponding to the diameter of the smallest hole through which sample will pass three tests out of three (equipment from Hanson Research Corporation Chatsworth).

The particle size determination is carried out by means of airjet sifting under the following conditions: individual sieves according to ASTM E11, 10 g of substance, the equipment used is the Alpine airjet sieve, a low pressure is used, preferably 250 mm H₂O (between 100-300 mm H₂O), the sieving period is 5 minutes, and the auxiliary is 0.30 g antistatic per 10 g substance and preferably Aerosil R 972 (Degussa).

In the tablet according to the invention the particle size of the pseudoephedrine present is chosen such that it has an ability to settle of less than 30 ml. The ability to settle (V₁₀-V₅₀₀) is measured according to Eur. Pharm. 2.9.15.

Preferably in the tablet according to the invention not more than 10% of the pseudoephedrine present therein has a particle size of less than 100-μm. More preferably the particle size of the pseudoephedrine is such that at least 95% of the particles are less than 500 μm and not more than 15% are less than 106 μm.

The best results have been obtained with a tablet wherein the pseudoephedrine is crystalline.

The tablet according to a preferred embodiment of the invention comprises, as hydrophilic polymer, a methyl cellulose ether derivative and preferably a substituted hydroxylated methyl cellulose.

The viscosity of the methyl cellulose ether derivative is measured according to Eur. Pharm. described method in cellulose derivatives monographs or according to USP method no <911>.

The best results have been obtained with the product sold under the trademark Methocel K15 MCR, which is an hydroxypropylmethylcellulose (methoxyl: 19-24%, hydroxypropyl: 7-12%), chlorides: max 0.5%; having an apparent viscosity of 11000 to 21000 mPa (=cP) and a particle size: min 90%<100 mesh.

Preferably the ratio of hydroxypropylmethylcellulose (HPMC) to the pseudoephedrine is between 0.5 to 2 by weight.

In the tablet according to a preferred embodiment of the invention the efletirizine containing segment also contains a disintegrant, preferably in the range less than 5% by weight of efletirizine segment and most preferably in the range of 1 to 5%. Examples of suitable disintegrant are sodium starch glycolate, sodium crosscarmelose (cross-linked carboxy methyl cellulose), polyvinylpyrrolidone derivatives, crospovidone (trademark Polyplasdone XL, PLP XL). The best results have been obtained with a disintegrant being a cross-linked carboxy methyl cellulose.

In a preferred embodiment of the tablet the efletirizine segment contains excipients including a polyhydroxyl compound having a molecular weight of less than 400. Preferably the polyhydroxyl compound is a sugar. Most preferably the sugar is lactose.

A more preferred embodiment of the invention is the tablet which is a bi-layer tablet, the efletirizine segment being a layer and the pseudoephedrine segment being a layer. Preferably the weight ratio of the pseudoephedrine layer to the efletirizine layer is between 0.25 to 10, and most preferably between 2 and 6.

In the preferred embodiment the outer face of each of the two layers has a different shape. Preferably the tablet has a first face which is the pseudoephedrine layer, having multiple radii of curvature, and most preferably three. Preferably the tablet has a second face which is the efletirizine layer, having a single radius of curvature. Radius of curvature is defined in American Pharmaceutical Association (Tableting Specification Manual, 4^{th} edition, 2215 Constitution Avenue, NR, Washington, D.C. 20037-2985, pp 45 and 46); cup radius is a single arc generated from the tablet's centerline (midpoint) across the tablet's diameter, minor axis or major axis; the cup radius forms the cup's profile; cup is the depression, or concavity, at the end of a punch tip; Major axis: length of a shaped tablet, minor axis is width of a shaped tablet.

A tablet may comprise an additional coating layer. In an alternative the coating layer can act as a taste masking agent. Examples of suitable taste masking agents are cellulose derivatives (methyl-, carboxymethyl-hydroxymethyl-, hydroxy ethyl-, hydroxymethylpropyl, cellulose) vinyl derivatives (polyvinyl alcohol, polyvinyl acetate), acrylic and methacrylic derivatives (Eudragits®), maleic copolymers, polyoxyethylene glycols, natural resins (zeine, gums).

A tablet may also contain some pharmaceutically acceptable fillers as excipients. Examples of suitable fillers are starch and derivatives, lactose, mannitol, sucrose, glucose, sorbitol, calcium phosphates, maltodextrines, polyvinylpyrrolidone, polyethylene glycols, microcrystalline cellulose, organic acids.

In a preferred embodiment of the invention the tablet is packaged in a moisture and oxygen protective packaging material.

In a tablet according to a preferred embodiment of the invention, the pseudoephedrine segment comprises at least one excipient, selected from inert matrices, hydrophilic matrices, lipid matrices, mixtures of inert matrices and of lipid matrices, mixtures of hydrophilic matrices and of lipid matrices, mixtures of hydrophilic matrices and of inert matrices.

The tablets according to a preferred embodiment of the present invention comprise matrix excipients chosen from inert, hydrophilic and lipophilic matrices.

Examples of inert matrices which can be used according to the present invention are: polyvinyl chloride, polyethylene, vinyl acetate/vinyl chloride copolymers, polymethylmethacrylates, polyamides, silicones, ethyl cellulose, polystyrene and the like.

Examples of hydrophilic matrices which can be used according to the present invention are cellulose derivatives hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like) and acrylic acid polymers (carbopols 934P and 974P and the like). The hydrophilic matrices preferably used according to the present invention are hydroxypropyl methyl celluloses, such as compounds sold under the trademark METHOCEL K or E.

Examples of lipid matrices which can be used according to the present invention are glycerides (mono-, di- or triglycerides: stearin, palmitin, laurin, myristin, hydrogenated castor or cottonseed oils, precirol and the like), fatty acids and alcohols (stearic acid, palmitic acid, lauric acid; stearyl alcohol, cetyl alcohol, cetyl stearyl alcohols, and the like), fatty acid esters (monostearates of propylene glycol and of sucrose, sucrose distearate and the like) and waxes (white wax, cachalot wax and the like).

In addition to the above-mentioned components, the tablet according to the present invention may also contain other excipients such as diluents (example: Emcompress, lactose and the like), binders (Avicel, starches, polyvinylpyrrolidone and the like), disintegrants (starches and modified starches, cellulose derivatives, alginic derivatives, pectins and the like), lubricants (talc, magnesium stearate, colloidal silica and the like), taste-masking agents (α-cyclodextrin, β-cyclodextrin, γ-cyclodextrin and their alkylated derivatives), flavourings or colourings as well as coating agents (example: cellulose derivatives, methacrylic resins, polyvinyl chloride, nylons and the like).

For implementing the method of treatment of the invention the tablet hereinabove described should contain an effective amount of efletirizine and pseudoephedrine. An effective amount can be readily determined by the use of conventional techniques and by observing results obtained under analogous circumstance. In determining the effective amount, a number of factors are considered including, but not limited to: the species of patient; its size, age, and general health; the specific disease involved; the degree of or involvement or the severity of the disease; the response of the individual patient; the particular compound administered; the mode of administration; the bioavailability characteristics of the preparation administered; the dose regimen selected; and the use of concomitant medication.

Additionally, the respective proportions of efletirizine and pseudoephedrine in the tablet should preferably be such that the said tablet comprises about 0.25 to about 2.5 percent by weight of efletirizine and about 10 to about 45 percent by weight of pseudoephedrine.

A tablet according to the invention can be administered to a patient in any form or mode which makes the tablet bioavailable in effective amounts, namely the oral route. One skilled in the art of preparing formulations can readily select the proper form and mode of administration depending upon the particular characteristics of the disease state to be treated, the stage of the disease, and other relevant circumstances.

The tablets of the invention can comprise at least one pharmaceutically acceptable excipient, the proportion and nature of which are determined by the solubility and chemical properties of the tablet selected, the chosen route of administration, and standard pharmaceutical practice.

More particularly, the present invention contemplates pharmaceutical compositions consisting essentially of a therapeutically effective amount of the above-described active compounds in association with one or more pharmaceutically acceptable excipients.

The excipient material may be a solid or semi-solid material which can serve as a vehicle or medium for the active ingredient. Suitable excipient materials are well known in the art. The pharmaceutical tablets of the invention may be adapted for oral use and may be administered to the patient in the form of tablets, or capsules.

The excipient material should be suitably selected with respect to the intended form of administration, and consistent with conventional pharmaceutical practice. For instance, for oral administration in the form of tablets or capsules, the therapeutically active drug components may be combined with any oral non-toxic pharmaceutically acceptable inert excipient such as lactose or starch. Optionally, the pharmaceutical tablet of the invention also contain a binder such as microcrystalline cellulose, gum tragacanth or gelatine, a disintegrating agent such as alginic acid, a lubricant such as magnesium stearate, a glidant such as colloidal silicon dioxide, a sweetening agent such as sucrose or saccharin, a coloring agent or a flavouring agent such as peppermint or methyl salicylate

Because of their easy administration, tablets represent the most advantageous oral dosage unit form. If desired, tablets may be coated by standard aqueous or nonaqueous techniques with sugar, shellac or other coating agents, for example enteric coating agents. Desirably, each tablet or capsule contains from about 15 mg to about 300 mg of the active ingredients.

A tablet according to the invention can be prepared according to various methods known to persons skilled in the art.

The present invention concerns also the use of a tablet described, for the manufacture of a medicament for preventing or treating disorders or conditions associated with rhinitis, cold, flu, cold-like and flu-like symptoms and allergic rhinitis, relief of nasal congestion, seasonal rhinitis, sneezing, rhinorrhea, nasal and ocular pruritus, redness of the eyes, tearing, sneezing.

The present invention concerns also a method for preventing or treating in humans and mammals disorders or conditions associated with rhinitis, cold, flu, cold-like and flu-like symptoms and allergic rhinitis, relief of nasal congestion, seasonal rhinitis, sneezing, rhinorrhea, nasal and ocular pruritus, redness of the eyes, tearing, sneezing.

The invention is further defined by reference to the following examples describing in detail the tablets of the present invention, as well as their utility.

### EXAMPLE 1

### Composition of the Pseudoephedrine Slow Release Segment of the Bilayer Tablets

A phase one, opened, randomised pilot study compared the oral bioavailability of experimental 120 mg sustained release segment pseudoephedrine formulations (table 1).

TABLE 1
Composition of tablets A and B.
mg/tablet

| Components | A | B |
|---|---|---|
| Pseudoephedrine•HCl | 120 | 120 |
| HPMC (a) | — | 120 |
| HPMC (b) | 200 | — |
| Microcrystalline cellulose | 74 | 55.5 |
| Colloidal silicon dioxide | 2 | 1.5 |
| Magnesium stearate | 4 | 3 |

HPMC (a) represents a compound hydroxypropyl methylcellulose having an apparent viscosity of 11250 to 21000 mPA (=cP (centipoises)), as defined in USP monograph hydroxypropyl methylcellulose.

HPMC (b) represents a compound hydroxypropyl methylcellulose having an apparent viscosity of 80000 to 120000 mPa (=cP).

The objective was to compare the oral bioavailability of the experimental sustained release formulations and an immediate release reference tablet (60 mg) given twice a day in 8 healthy male subjects.

The main pharmacokinetic parameters are listed in table 2.

TABLE 2
Main pharmacokinetic parameters after oral administration
of 120 mg of pseudoephedrine in 8 healthy volunteers

| Treatment | Reference | A | B |
|---|---|---|---|
| Cₘₐₓ (ng/mL) | 391 | 259 | 295 |
| tₘₐₓ (h) | 1.5 | 5 | 5 |
| AUC (ng · h/mL) | 3877 | 3943 | 4249 |

The two experimental formulations (A and B), which showed a clear slow release profile, were bioequivalent to the reference formulation.

The B formulation was chosen for further development as pseudoephedrine layer given its longer plateau time in the curve compared to formulation A.

### EXAMPLE 2

### Dissolution Profile's pH Dependence for Tablet B Segment

Dissolution profile of pseudoephedrine is assessed at various pHs (water, HCl 0.1 N, pH 4.5, 6.8 and 7.5, USP 24 Apparatus 1, 100 rpm, 37° C.). Results are ex-pressed in table 3.

TABLE 3
In vitro dissolution data of tablet B segment at various pHs.

| Time (h) | Water | HCl 0.1 N | pH 4.5 | pH 6.8 | pH 7.5 |
|---|---|---|---|---|---|
| 0 | — | — | — | — | — |
| 1 | 44.1 | 39.4 | 39.6 | 40.4 | 41.4 |
| 2 | 62.1 | 57.0 | 58.0 | 58.2 | 59.4 |
| 3 | 74.9 | 68.5 | 70.1 | 70.8 | 70.5 |
| 4 | 84.0 | 77.9 | 79.0 | 79.6 | 79.2 |
| 6 | 92.7 | 89.3 | 92.3 | 90.8 | 90.6 |
| 8 | 97.8 | 97.3 | 96.7 | 96.7 | 96.2 |
| 12 | — | 105.3 | 101.2 | 100.9 | 99.9 |

The results show a pH-independent in vitro dissolution.

### EXAMPLE 3

### Composition Efletirizine.HCl/Pseudoephedrine.HCl 10 mg/120 mg Bi-Layer Tablet

Coated efletirizine.HCl/pseudoephedrine.HCl bi-layer tablets were prepared.

The particle size of the pseudoephedrine is such that at least 95% of the particles are less than 500 μm and not more than 15% are less than 106 μm.

The formulation of these tablets is presented in table 4.

TABLE 4
Composition of 10 mg/120 mg film
coated efletirizine•HCl/pseudoephedrine•HCl
tablets

| | | mg/tablet |
|---|---|---|
| Core's first layer: | Pseudoephedrine•HCl | 120 |
| | HPMC (a) | 120 |
| | Microcrystalline cellulose | 57 |
| | Colloidal silicon dioxide | 1.5 |
| | Magnesium stearate | 1.5 |
| Core's second layer: | Efletirizine•HCl | 10.00 |
| | Lactose monohydrate | 91.20 |
| | Microcrystalline cellulose | 50.00 |
| | Croscarmellose sodium | 6.40 |
| | Colloidal silicon dioxide | 0.80 |
| | Magnesium stearate | 1.60 |
| Coating material: | Opadry white | 13.80 |

The product Opadry white is a combination of polymers for the aqueous film coating (hydroxypropylmethylcellulose, titanium dioxide, polyethylene glycol 400).

The components of each core layer are mixed separately an then compressed in a bi-layer rotary tablet press. Then the tablets are coated with Opadry.

The tablet has a first face, which is the pseudoephedrine layer, having multiple radii of curvature. The tablet has a second face, which is the efletirizine layer, having a single radius of curvature.

The interfacial surface area of the pseudoephedrine segment and efletirizine segment is about 95 mm². The diameter of the tablet is about 11 mm.

The tablet is packaged in a moisture and oxygen protective packaging material.

1. A tablet comprising at least two distinct segments, one segment of which comprises as active ingredient predominantly efletirizine and a second segment of which comprises as active ingredient predominantly pseudoephedrine, said segments being composed and formed in such a way that the resulting tablet is substantially free of impurities formed by reaction of efletirizine with pseudoephedrine, and with the proviso that the tablet comprises less than 5% by weight, relative to the total weight of the pseudoephedrine segment, of an alkalinizing agent.

2. A tablet comprising at least two distinct segments one segment of which comprises as active ingredient predominantly efletirizine and a second segment of which comprises as active ingredient predominantly pseudoephedrine, said segments being composed and formed in such a way that the pharmacokinetic profiles of the efletirizine and pseudoephedrine are substantially the same as in a dosage form containing each as sole active ingredient in the same amount.

3. A tablet according to claim 1 or 2 wherein the pseudoephedrine segment is substantially free of efletirizine.

4. A tablet according to claim 1 or 2 wherein the efletirizine segment is substantially free of pseudoephedrine.

5. A tablet according to any one of the preceding claims wherein the interfacial surface area of the pseudoephedrine segment and efletirizine segment is less than 180 mm².

6. A tablet according to any one of the preceding claims wherein the tablet further comprises a barrier segment wherein said barrier segment separates the efletirizine segment and the pseudoephedrine segment.

7. A tablet according to any one of the preceding claims wherein the pseudoephedrine segment comprises less than 5% by weight, relative to the total weight of the pseudoephedrine segment, of an alkalinizing agent.

8. A tablet according to any one of the preceding claims wherein the tablet comprises a plurality of pseudoephedrine segments.

9. A tablet according to any one of the preceding claims wherein said efletirizine segment is in the form of a compression coating.

10. A tablet according to any one of the preceding claims wherein said efletirizine segment is in the form of a spray coating.

11. A tablet according to any one of the preceding claims wherein the pseudoephedrine segment contains inert pharmaceutical excipients in an amount of 0.75 to 4.5 times that of the pseudoephedrine itself by weight.

12. A tablet according to any one of the preceding claims wherein the efletirizine segment contains inert pharmaceutical excipients in an amount of 5 to 30 times that of the efletirizine itself by weight.

13. A tablet according to any one of the preceding claims wherein the ratio of the total amount of inert pharmaceutical excipients present to the total aggregate amount of all active ingredients is between 1.2 and 6 by weight.

14. A tablet according to any one of the preceding claims wherein the weight ratio of pseudoephedrine to efletirizine is between 2 and 40.

15. A tablet according to claim 14 wherein the weight ratio of pseudoephedrine to efletirizine is about 12.

16. A tablet according to any one of the preceding claims wherein the pseudoephedrine segment comprises between about 10 and 265 mg of pseudoephedrine and the efletirizine segment comprises between about 3 and 70 mg of efletirizine.

17. A tablet according to any one of the preceding claims wherein the pseudoephedrine segment is in a slow release form.

18. A tablet according to any one of the preceding claims wherein the efletirizine is in an immediate release form.

19. A tablet according to any one of the preceding claims wherein the tablet weight is between 200 to 800 mg.

20. A tablet according to any one of the preceding claims wherein the tablet comprises an amount of efletirizine which when dosed to a human subject gives a efletirizine area under the plasma efletirizine concentration versus time curve which is between 80% and 125% of the area under the plasma efletirizine concentration versus time curve observed when a dihydrochloride efletirizine immediate release tablet comprising said amount of efletirizine is dosed to same human subject at the same efletirizine dose.

21. A tablet according to any one of the preceding claims wherein the tablet comprises an amount of pseudoephedrine which when dosed to a human subject gives a pseudoephedrine area under the pseudoephedrine plasma concentration versus time curve which is between 80% and 125% of the area under the plasma pseudoephedrine concentration versus time curve observed when a pseudoephedrine sustained release tablet comprising said amount of pseudoephedrine is dosed to same human subject.

22. A tablet according to any one of the preceding claims wherein the particle size of the pseudoephedrine present is chosen such that it has a flow index less than 25.

23. A tablet according to any one of the preceding claims wherein the particle size of the pseudoephedrine present is chosen such that it has an ability to settle of less than 30 ml.

24. A tablet according to any one of the preceding claims wherein not more than 10% of the pseudoephedrine present therein has a particle size of less than 100 μm.

25. A tablet according to claim 23 or 24 wherein the particle size of the pseudoephedrine is such that at least 95% of the particles are less than 500 μm and not more than 15% are less than 106 μm.

26. A tablet according to any one of claims 23 to 25 wherein the pseudoephedrine is crystalline.

27. A tablet according to any one of the preceding claims wherein the pseudoephedrine containing segment also contains a methyl cellulose ether derivative having a viscosity of about 11,000 to 21,000 mPa.

28. A tablet according to claim 27 wherein the methyl cellulose ether derivative is a substituted hydroxylated methyl cellulose.

29. A tablet according to claim 27 wherein the methyl cellulose ether derivative is an hydroxypropylmethylcellulose.

30. A tablet according to claim 29 wherein the derivative is an hydroxypropylmethylcellulose (methoxyl: 19-24%, hydroxypropyl: 7-12%), chlorides: max 0.5%; having an apparent viscosity of 11250 to 21000 mPa and a particle size: min 90%<100 mesh.

31. A tablet according to any one of claims 27 to 30 wherein the ratio of hydroxypropylmethylcellulose (HPMC) to the pseudoephedrine is between 0.5 to 2 by weight.

32. A tablet according to any one of the preceding claims wherein the efletirizine containing segment also contains a disintegrant.

33. A tablet according to claim 32 wherein the efletirizine containing segment also contains a disintegrant in the range less than 5% by weight of efletirizine segment.

34. A tablet according to claim 32 wherein the disintegrant is a cross-linked carboxy methyl cellulose.

35. A tablet according to any one of the preceding claims wherein the efletirizine segment contains excipients including a polyhydroxyl compound having a molecular weight of less than 400.

36. A tablet according to claim 35 wherein the polyhydroxyl compound is a sugar.

37. A tablet according to claim 36 wherein the sugar is lactose.

38. A tablet according to any one of the preceding claims wherein the tablet is a bi-layer tablet, the efletirizine segment being a layer and the pseudoephedrine segment being a layer.

39. A tablet according to claim 38 wherein the weight ratio of the pseudoephedrine layer to the efletirizine layer is between 0.25 to 10.

40. A tablet according to claims 38 or 39 wherein the outer face of each of the two layers has a different shape.

41. A tablet according to claim 40 wherein the tablet has a first face which is the pseudoephedrine layer, having multiple radii of curvature.

42. A tablet according to claim 40 wherein the tablet has a second face which is the efletirizine layer, having a single radius of curvature.

43. A tablet according to anyone of the preceding claims which comprises an additional coating layer.

44. A tablet according to claim 43 wherein the coating layer can act as a taste masking agent.

45. A tablet according to anyone of the preceding claims wherein the tablet is packaged in a moisture protective packaging material.

46. A tablet according to anyone of the preceding claims wherein the tablet is packaged in an oxygen protective packaging material.

47. A tablet according to anyone of the preceding claims wherein the efletirizine segment comprises efletirizine dihydrochloride.

48. Use of a tablet according to anyone of the preceding claims, for the manufacture of a medicament for preventing or treating disorders or conditions associated with rhinitis, cold, flu, cold-like and flu-like symptoms, and allergic rhinitis, relief of nasal congestion, seasonal rhinitis, sneezing, rhinorrhea, nasal and ocular pruritus, redness of the eyes, tearing, sneezing.
